(19) United States
(12) Patent Application Publication
(10) Pub. No.: US 2020/0246594 A1
(43) Pub. Date: Aug. 6, 2020

(54) Catheter Discharge Apparatus

(71) Applicant: Viola Miller, Vancouver (CA)

(72) Inventor: Viola Miller, Vancouver (CA)

(21) Appl. No.: 16/264,081

(22) Filed: Jan. 31, 2019

(51) Int. Cl. A61M 25/04; A61M 25/00

(52) U.S. Cl. CPC A61M 25/04; A61M 25/0017; A61M 39/22; A61M 25/0075; A61M 25/0041

(57) ABSTRACT

A catheter discharge apparatus that is configured to operably couple to an end of a catheter tube wherein the catheter discharge apparatus inhibits the flow or urine therethrough in order to improve bladder function. The catheter discharge apparatus includes a body having a first portion and a second portion that are both tubular in shape having a hollow passage therethrough. A valve is present intermediate the first portion and second portion of the body and has a first position and second position configured to inhibit or allow flow from the first portion of the body into the second portion of the body. The valve has a lever operably coupled thereto, which is configured with an engagement member that is angular in manner with respect to the first portion of the body when the lever is in its first position. The first portion has a plurality of securing rings formed thereon.

### FIELD OF THE INVENTION

The present invention relates generally to medical apparatus, more specifically but not by way of limitation, a discharge apparatus that is configured to be releasably secured to a urethral catheter wherein the apparatus is configured to be operable with a single hand and wherein the apparatus of the present invention assists in increasing the bladder capacity and bladder muscle of the user.

### BACKGROUND

Urethral catheterization is a procedure that is routinely performed on both men and women. Urethral catheterization facilitates direct drainage of the bladder of the patient and can be installed on patients for many reasons such as but not limited to post-surgery, diagnostic reasons or urinary retention issues. As is known in the art, during installation of the catheter, the catheter tube is inserted into the urethra until the end enters the bladder and subsequently urine will begin to flow from the catheter. A typical installation of a catheter will require a bag at the opposing end of the catheter tube wherein the bag is utilized to collect urine. Collection of the urine may be necessary for testing but it can often be untested and subsequently removed once the bag has been filled to near capacity.

As is known in the art, the wearing of a catheter bag is considered inconvenient by most patients. For those patients that must have a catheter installed for longer periods of time, the management of the catheter bag can be cumbersome as some wardrobe items are difficult to wear and provide concealment of the catheter bag. Additionally, while the patient has a catheter tube installed the patient's bladder and muscular function thereof is not in use. Since the catheter tube is installed into the bladder, any urine present in the bladder is drained into the bag. As a result, the bladder muscle becomes atrophied and the unused thereof can result in overall less effective bladder control for the patient. As many patients desire to eliminate their need for a catheter or having to carry a catheter bag, elimination of the catheter bag is desirable.

Accordingly, there is a need for a catheter discharge apparatus that is configured to operably couple to a catheter tube distal to the bladder end of the catheter tube wherein the apparatus inhibits the flow of urine out from the tube and has a body size so as to facilitate the operation thereof with a single hand of the user.

### SUMMARY OF THE INVENTION

It is the object of the present invention to provide a catheter discharge apparatus that is configured to be secured to a catheter tube on an end distal to the end of the catheter tube disposed within the bladder wherein the catheter apparatus of the present invention includes a body having a first portion and a second portion.

Another object of the present invention is to provide a catheter discharge apparatus that is configured to assist a patient in the strengthening of their bladder function wherein the first portion of the body is tubular in shape and includes a hollow passage therethrough.

A further object of the present invention is to provide a catheter discharge apparatus that is configured to be secured to a catheter tube on an end distal to the end of the catheter tube disposed within the bladder wherein the first portion of the body is configured to be releasably secured to an end of the catheter tube.

Still another object of the present invention is to provide a catheter discharge apparatus that is configured to assist a patient in the strengthening of their bladder function wherein the first end of the body includes a cap releasably secured thereto.

An additional object of the present invention is to provide a catheter discharge apparatus that is configured to be secured to a catheter tube on an end distal to the end of the catheter tube disposed within the bladder wherein the body further includes a valve intermediate the first portion and second portion.

Yet a further object of the present invention is to provide a catheter discharge apparatus that is configured to assist a patient in the strengthening of their bladder function wherein the valve includes a lever that is rotatably coupled to the body and is operable to move the valve intermediate its first position and second position.

Another object of the present invention is to provide a catheter discharge apparatus that is configured to be secured to a catheter tube on an end distal to the end of the catheter tube disposed within the bladder wherein the second portion of the body includes a hollow passage therethrough and is fluidly coupled to the valve.

Still another object of the present invention is to provide a catheter discharge apparatus that is configured to assist a patient in the strengthening of their bladder function wherein the second portion of the body includes a discharge port that is configured to provide discharge of urine therethrough.

To the accomplishment of the above and related objects the present invention may be embodied in the form illustrated in the accompanying drawings. Attention is called to the fact that the drawings are illustrative only. Variations are contemplated as being a part of the present invention, limited only by the scope of the claims.

### BRIEF DESCRIPTION OF THE DRAWINGS

A more complete understanding of the present invention may be had by reference to the following Detailed Description and appended claims when taken in conjunction with the accompanying Drawings wherein:

FIG. 1 is a perspective view of a preferred embodiment of the present invention.

### DETAILED DESCRIPTION

Referring now to the drawings submitted herewith, wherein various elements depicted therein are not necessarily drawn to scale and wherein through the views and figures like elements are referenced with identical reference numerals, there is illustrated a catheter discharge apparatus 100 constructed according to the principles of the present invention.

An embodiment of the present invention is discussed herein with reference to the figures submitted herewith. Those skilled in the art will understand that the detailed description herein with respect to these figures is for explanatory purposes and that it is contemplated within the scope of the present invention that alternative embodiments are plausible. By way of example but not by way of limitation, those having skill in the art in light of the present teachings of the present invention will recognize a plurality of alternate and suitable approaches dependent upon the needs of the particular application to implement the functionality of any given detail described herein, beyond that of the particular implementation choices in the embodiment described herein. Various modifications and embodiments are within the scope of the present invention.

It is to be further understood that the present invention is not limited to the particular methodology, materials, uses and applications described herein, as these may vary. Furthermore, it is also to be understood that the terminology used herein is used for the purpose of describing particular embodiments only, and is not intended to limit the scope of the present invention. It must be noted that as used herein and in the claims, the singular forms “a”, “an” and “the” include the plural reference unless the context clearly dictates otherwise. Thus, for example, a reference to “an element” is a reference to one or more elements and includes equivalents thereof known to those skilled in the art. All conjunctions used are to be understood in the most inclusive sense possible. Thus, the word “or” should be understood as having the definition of a logical “or” rather than that of a logical “exclusive or” unless the context clearly necessitates otherwise. Structures described herein are to be understood also to refer to functional equivalents of such structures. Language that may be construed to express approximation should be so understood unless the context clearly dictates otherwise.

References to “one embodiment”, “an embodiment”, “exemplary embodiments”, and the like may indicate that the embodiment(s) of the invention so described may include a particular feature, structure or characteristic, but not every embodiment necessarily includes the particular feature, structure or characteristic.

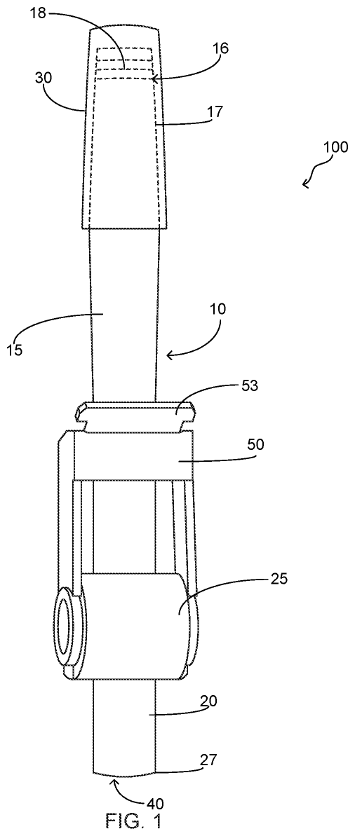

Referring in particular to FIG. 1, the catheter discharge apparatus 100 includes a body 10. The body 10 is manufactured from a suitable durable material such as but not limited to plastic. It is desirable within the scope of the present invention that the body 10 be manufactured from a material that facilitates the cleaning and re-use thereof. Those skilled in the art will recognize that numerous types of material could be utilized to manufacture the body 10. The body 10 includes a first portion 15, a valve 25 and a second portion 20 integrally formed utilizing suitable techniques. The first portion of the body 10 is tubular in shape and has a hollow passage therethrough (not particularly illustrated herein). The first portion 15 includes end 16 wherein end 16 is distally located from valve 25.

End 16 is slightly tapered in formed wherein the tapered form of the end 16 promotes easy coupling with the end of a conventional catheter tube. The end 16 includes a plurality of securing rings 18 formed thereon. The securing rings 18 are formed on the end 16 specifically on the outer surface thereof utilizing suitable techniques. The securing rings 18 on the end 16 are raised with respect to the outer surface 17 of the end 16 and are operable to provide a more secure and leak-proof coupling with the end of a conventional catheter tube. While two securing grooves 18 are illustrated herein, it is contemplated within the scope of the present invention that the catheter discharge apparatus 100 could have more or less than two securing rings 18 formed on the end 16 thereof.

Releasably secured to the first portion 15 specifically the end 16 thereof is cap 30. The cap 30 is manufactured from a pliable material such as but not limited to silicon and is configured to be releasably secured to the end 16 of the first portion 15. The cap 30 is protective in function wherein the cap 30 is in place until a user of the catheter discharge apparatus 100 is ready to attach the catheter discharge apparatus 100 to an end of a catheter at which time the cap 30 is removed. It is contemplated within the scope of the present invention that the cap 30 could be formed in alternate shapes and/or styles and still achieve the desired functionality as described herein.

Integrally formed with the first portion 15 distal to end 16 is valve 25. Valve 25 is configured to have a first position and a second position wherein in the first position the valve 25 inhibits flow of urine into the second portion 20 of the body 10. In the second position of the valve 25 the valve is in an open position so as to allow the flow of urine into the second portion 20 of the body 10 and out the discharge port 40. The lever 50 is operably coupled to the valve 25 and is configured to move the valve 25 intermediate the aforementioned first position and second position. As illustrated herein, the lever 50 is in its first position wherein the lever 50 is adjacent the body 10 and as such this corresponds to the first position of the valve 25 wherein the valve 25 is closed inhibiting flow of urine therethrough. The lever 50 is manufactured from a suitable durable material such as but not limited to plastic. The lever 50 is formed so as to have engagement member 53 wherein the engagement member 53 is angular in manner wherein the angle position thereof provides improved ability for a user to operate the lever 50 with one hand.

The aforementioned position of the lever 50 in its first position is desirable as this reduces any interference with clothing or the like when a patient is utilizing the catheter discharge apparatus 100 while dressed in pants or similar type clothing. The lever 50 is moved to its second position wherein the lever 50 is perpendicular to the body 10 and in this position the valve 25 is moved to its second position and urine is then permitted to flow into the second portion 20 and out the discharge port 40 into a suitable location.

The second portion 20 of the body 10 is integrally formed with the valve 25 and includes a hollow passage (not illustrated herein) therethrough. The discharge port 40 is located and the end 27 of the second portion 20 distal to the valve 25. The discharge port 40 and second portion 20 combine to assist the user of the catheter discharge apparatus 100 to direct a flow of urine stream into a desired container. It is contemplated within the scope of the present invention that the second portion 20 could be formed in alternate lengths.

In the preceding detailed description, reference has been made to the accompanying drawings that form a part hereof, and in which are shown by way of illustration specific embodiments in which the invention may be practiced. These embodiments, and certain variants thereof, have been described in sufficient detail to enable those skilled in the art to practice the invention. It is to be understood that other suitable embodiments may be utilized and that logical changes may be made without departing from the spirit or scope of the invention. The description may omit certain information known to those skilled in the art. The preceding detailed description is, therefore, not intended to be limited to the specific forms set forth herein, but on the contrary, it is intended to cover such alternatives, modifications, and equivalents, as can be reasonably included within the spirit and scope of the appended claims.

What is claimed is:

1. A catheter discharge apparatus comprising:
a body, said body having a first portion and a second portion integrally formed, said first portion having a hollow passage therethrough, said second portion of said body having a hollow passage therethrough, said first portion of said body configured to be coupled to a catheter tube;
a valve, said valve being intermediate said first portion and said second portion of said body, said valve having a first position and a second position, said first position of said valve said valve is in a closed position inhibiting the flow of fluid therethrough;
a lever, said lever being rotatably coupled to said body, said lever operably coupled to said valve, said lever configured to provide a user ability to move said valve intermediate said first position and said second position wherein said lever includes a first position and second position corresponding with said valve.

2. The catheter discharge apparatus as recited in claim 1, wherein said first portion of said body further includes an end, said end being distal to said valve, said end being tapered in shape.

3. The catheter discharge apparatus as recited in claim 2, and further including at least one securing ring, said at least one securing ring being formed on said end of said first portion, said at least one securing ring operable to provide an improved coupling with a catheter tube.

4. The catheter discharge apparatus as recited in claim 3, wherein in said first position said lever is adjacent said body.

5. The catheter discharge apparatus as recited in claim 4, wherein said second portion of said body includes a discharge port, said discharge port distal to said valve, said discharge port configured to direct fluid out from said body.

6. The catheter discharge apparatus as recited in claim 5, wherein in the second position said lever is perpendicular to said body.

7. The catheter discharge apparatus as recited in claim 6, and further including an engagement member, said engagement member being formed on said lever, said engagement member being angular in manner to said first portion of said body.

8. A catheter discharge apparatus configured to provide a patient improved bladder functionality and reduce utilization of a catheter bag wherein the catheter discharge apparatus comprises:
a body, said body having a first portion and a second portion integrally formed, said first portion having a hollow passage therethrough, said second portion of said body having a hollow passage therethrough, said first portion of said body having an end configured to be coupled to a catheter tube, said end of said first portion being tapered in form;
a valve, said valve fluidly coupled to said first portion and said second portion of said body, said valve being intermediate said first portion and said second portion of said body, said valve having a first position and a second position, wherein in said first position of said valve said valve is in a closed position inhibiting the flow of fluid from said first portion to said second portion of said body;
a lever, said lever being rotatably coupled to said body, said lever operably coupled to said valve, said lever configured to provide a user ability to move said valve intermediate said first position and said second position wherein said lever includes a first position and second position corresponding with said valve.

9. The catheter discharge apparatus as recited in claim 8, and further including a cap, said cap being configured to be releasably secured to said end of said first portion when the catheter discharge apparatus is not in use.

10. The catheter discharge apparatus as recited in claim 9, wherein said end of said first portion further includes a plurality of securing rings formed thereon, said securing rings being formed on the outer surface of said end of said first portion, said securing rings being raised in form.

11. The catheter discharge apparatus as recited in claim 10, and further including an engagement member, said engagement member being formed on said lever, said engagement member being angular in manner to said first portion of said body.

12. The catheter discharge apparatus as recited in claim 11, wherein said second portion of said body includes a discharge port, said discharge port distal to said valve, said discharge port configured to direct fluid out from said body.

13. The catheter discharge apparatus as recited in claim 12, wherein in the second position said lever is perpendicular to said body.

14. The catheter discharge apparatus as recited in claim 13, wherein in said first position said lever is adjacent said body.

15. A catheter discharge apparatus configured to provide a patient improved bladder functionality and reduce utilization of a catheter bag wherein the catheter discharge apparatus comprises:
a body, said body having a first portion and a second portion integrally formed, said first portion having a hollow passage therethrough, said second portion of said body having a hollow passage therethrough, said first portion of said body having an end configured to be coupled to a catheter tube, said end of said first portion being tapered in form, wherein said second portion of said body includes a discharge port, said discharge port distal to said valve, said discharge port configured to direct fluid out from said body into a suitable container;
a valve, said valve fluidly coupled to said first portion and said second portion of said body, said valve being intermediate said first portion and said second portion of said body, said valve having a first position and a second position, wherein in said first position of said valve said valve is in a closed position inhibiting the flow of fluid from said first portion to said second portion of said body;
a lever, said lever being rotatably coupled to said body, said lever operably coupled to said valve, said lever configured to provide a user ability to move said valve intermediate said first position and said second position wherein said lever includes a first position and second position corresponding with said valve and wherein said lever further has an engagement member integrally formed as a part thereof, said engagement member being formed on said lever, said engagement member being angular in manner to said first portion of said body.

16. The catheter discharge apparatus as recited in claim 15, wherein said end of said first portion further includes a plurality of securing rings formed thereon, said securing rings being formed on the outer surface of said end of said first portion, said securing rings being raised with respect to said outer surface of said end.

17. The catheter discharge apparatus as recited in claim 16, wherein in the second position said lever is perpendicular to said body.

18. The catheter discharge apparatus as recited in claim 17, wherein in said first position said lever is adjacent said body.

19. The catheter discharge apparatus as recited in claim 18, wherein said body is manufactured from plastic.

20. The catheter discharge apparatus as recited in claim 19, and further including a cap, said cap being configured to be releasably secured to said end of said first portion when the catheter discharge apparatus is not in use.
